(19) United States
(12) Patent Application Publication
(10) Pub. No.: US 2025/0311912 A1
(43) Pub. Date: Oct. 9, 2025

(54) SYSTEMS AND METHODS FOR ENDOSCOPE LOCALIZATION

(71) Applicant: Noah Medical Corporation, San Carlos, CA (US)

(72) Inventors: Xiang ZHANG, Vienna, VA (US); Tao ZHAO, Sunnyvale, CA (US)

(21) Appl. No.: 19/205,483

(22) Filed: May 12, 2025

Related U.S. Application Data
(63) Continuation of application No. PCT/US2023/085703, filed on Dec. 22, 2023.
(60) Provisional application No. 63/477,890, filed on Dec. 30, 2022.

(51) Int. Cl. A61B 1/00; A61B 1/267; A61B 5/06; A61B 6/00; A61B 6/03

(52) U.S. Cl. CPC A61B 1/00009; A61B 1/2676; A61B 5/062; A61B 6/032; A61B 6/5247

(57) ABSTRACT

Methods of localizing a scope tip of an endoscope in an organ of a patient are provided. The method comprises: (a) obtaining (i) a biological model of an organ of a patient and (ii) electromagnetic (EM) data that is generated by an endoscope in the organ; (b) generating a plurality of localization hypotheses for the scope tip based on the biological model and the EM data; (c) generating a plurality of deformations, wherein each deformation of the plurality of deformations respectively maps each of the plurality of localization hypotheses for the scope tip to the EM data; (d) determining a localization for the scope tip from the plurality of localization hypotheses for the scope tip, wherein the localization corresponds to a predicted deformation of the plurality of deformations that satisfies a threshold; and (e) causing the localization for the scope tip to be presented on a graphical display.

### CROSS-REFERENCE

This application is a continuation of International Application No. PCT/US2023/085703, filed Dec. 22, 2023, which claims priority to U.S. Provisional Patent Application No. 63/477,890, filed on Dec. 30, 2022, which is entirely incorporated herein by reference.

### BACKGROUND

Early diagnosis of lung cancer is critical. Lung cancer remains the deadliest form of cancer with over 150,000 deaths per year. Compared to CT guided TTNA (CT-TTNA), navigational bronchoscopy has a better safety profile (less risk of pneumothorax, life threatening bleeding and length of stay) and the ability to stage the mediastinum but is associated with a lower diagnostic yield. The endoscopy (e.g., bronchoscopy) may involve accessing and visualizing the inside of a patient's lumen (e.g., airways) for diagnostic or therapeutic purposes. During a procedure, a flexible tubular tool such as, for example, an endoscope, may be inserted into the patient's body and an instrument can be passed through the endoscope to a tissue site identified for diagnosis or treatment.

Robotic bronchoscopy systems have gained interest for the biopsy of peripheral lung lesions. Robotic platforms offer superior stability, distal articulation, and visualization over traditional pre-curved catheters. Some of the traditional robotic bronchoscopy systems utilize shape sensing technology (SS) for guidance. SS catheters may have an embedded fiberoptic sensor that measures the shape of the catheter several hundred times a minute. Other traditional robotic bronchoscopy systems incorporate direct visualization, optical pattern recognition and geopositional sensing (OPRGPS) for guidance. Both SS and OPRGPS systems utilize a pre-planning CT scan to create an electronically generated virtual target. However, SS and OPRGPS systems can be prone to CT-to-body divergence (CT2BD). CT2BD is the discrepancy of the electronic virtual target and the actual anatomic location of the peripheral lung lesion. CT2BD can occur for a variety of reasons including atelectasis, neuromuscular weakness due to anesthesia, tissue distortion from the catheter system, bleeding, ferromagnetic interference, and perturbations in anatomy such as pleural effusions. Neither the SS system nor the OPRGPS platform has intra-operative real time correction for CT2BD. The combination of C2TBD and other forms of deformation, such as breathing motion of the lung, affect accuracy of the localization of the electromagnetic sensors in the endoscopic tip.

The previously existing solutions for localization, e.g., closest point searching and Bayes filtering, are based on rigid lung models that do not account for the deformation. Not accounting for the deformation may increase the length of the procedure, frustrate the operator, and ultimately lead a nondiagnostic procedure. Additionally, the location measurements obtained by existing methods may not be accurate due to noise such as Gaussian noise and bias due to tissue deformation.

### SUMMARY

Provided herein are systems, methods, computer-readable media, and techniques for localizing a scope tip of an endoscope in an organ of a patient that accounts for the deformation, CT2BD and breathing motion. Better scope tip localization results are achievable when the deformations are properly accounted for. In some cases, the systems, the methods, the computer-readable media, and the techniques to generate a prediction of one or both of (i) the current position and orientation of the scope tip in the patient frame or (ii) the path of the scope tip through the patient in the patient frame. The prediction may be utilized to guide (e.g., via the user) or drive the scope towards a preoperatively planned target location and aim the scope at the target.

In some cases, localization may be performed by obtaining electromagnetic (EM) data from an EM sensor system, registering the EM data to the CT scan (patient data) coordinates, and computing the scope tip position in the airways of the patient lung utilizing the EM sensor data. Based on the localization, information such as the distance to a lesion may be determined. However, traditional method maps a point location based on EM data to a point of the airway model based on CT scan (e.g., closest point) which may result in less accurate location result due to noise in the EM sensor data (e.g., Gaussian noise) and tissue deformation bias. The localization method herein beneficially improves the localization accuracy by treating the airway path as a deformable object (instead of rigid model as the traditional method) and identifying a best fitting deformable path from a plurality of hypothesis deformable paths that best matches to an EM data based path (instead of using conditional probability estimation to find the best match hypothesis point). The algorithm herein advantageously avoids Gaussian noise in the EM sensor data by finding the best fitting path (instead of point) and eliminates the error introduced by tissue bias by treating the airway as a deformable object. In some embodiments, the methods herein may identify the best fitting deformable path to determine the location of the endoscope tip by solving a non-linear optimization problem.

In some cases, the systems, the methods, the computer-readable media, and the techniques may be implemented alongside tomosynthesis. Tomosynthesis (may also be referred to as “tomo”) is limited angle tomography in contrast to full-angle (e.g., 180-degree tomography). However, tomosynthesis reconstruction does not have uniform resolution. For instance, resolution is often the poorest in the depth direction. The standard way to show a 3D volume dataset by three orthogonal planes (e.g., axial, sagittal and coronal) may be ineffective since two of the planes have poorer resolution. A common way to view tomosynthesis volume is to scroll in the depth direction where each slice has good resolution. In the case of pulmonology, it is viewed in the coronal plane and goes through the anterior-posterior (AP) direction by scrolling. Yet this has caused difficulty in determining the spatial relationship of the structures in the depth direction. It can be challenging to determine whether a tool (e.g., biopsy needle) is inside a lesion in the AP direction of a chest tomosynthesis reconstruction.

A need exists for methods and systems capable of localization of an endoscopic tip (e.g., determining whether a tool is within a target, such as a lesion) with improved accuracy and correctness. The present disclosure addresses the above needs by providing the systems, the methods, the computer-readable media, and the techniques for localizing a scope tip of an endoscope in an organ of a patient. In some cases, systems, methods, and computer-readable media may implement operations including: (a) obtaining (i) a biological model of an organ of a patient and (ii) electromagnetic (EM) data that is generated by an endoscope in the organ; (b) generating a plurality of localization hypotheses for the scope tip based on the biological model and the EM data; (c) generating a plurality of deformations, wherein each deformation of the plurality of deformations respectively maps each of the plurality of localization hypotheses for the scope tip to the EM data; (d) determining a localization for the scope tip from the plurality of localization hypotheses for the scope tip, wherein the localization corresponds to a predicted deformation of the plurality of deformations that satisfies a threshold; and (e) causing the localization for the scope tip to be presented on a graphical display.

In an aspect, a method for localizing an endoscope in a body part of a patient is provided. The method comprises: (a) obtaining a sequence of electromagnetic (EM) data; (b) generating an EM data-based path based at least in part on the sequence of the EM data; (c) identifying one or more path hypotheses based at least in part on a data point from the sequence of EM data, wherein the one or more path hypotheses have a shape based on a biological model of the body part of the patient; (d) generating one or more deformed paths by mapping the one or more path hypotheses to the EM data-based path using an optimization algorithm; and (e) selecting a deformed path from the one or more deformed paths based at least in part on a probability associated with each of the one or more path hypotheses and determining a location for a tip of the endoscope based on the selected deformed path.

In a separated yet related aspect, A system for localizing an endoscope in a body part of a patient, the system comprising: a memory storing computer-executable instructions; one or more processors in communication with the endoscope and configured to execute the computer-executable instructions to: (a) obtain a sequence of electromagnetic (EM) data; (b) generate an EM data-based path based at least in part on the sequence of the EM data; (c) identify one or more path hypotheses based at least in part on a data point from the sequence of EM data, wherein the one or more path hypotheses have a shape based on a biological model of the body part of the patient; (d) generate one or more deformed paths by mapping the one or more path hypotheses to the EM data-based path using an optimization algorithm; and (e) select a deformed path from the one or more deformed paths based at least in part on a probability associated with each of the one or more path hypotheses and determining a location for a tip of the endoscope based on the selected deformed path.

In some embodiments, prior to (c), the EM data-based path is translated into a coordinate frame of the biological model. In some embodiments, the biological model comprises one or more airways. In some cases, the EM data are acquired while navigating the tip of the endoscope along the one or more airways forward or backward.

In some embodiments, the EM data-based path is generated by applying binned filtering and/or time-wise filtering to the EM data. In some cases, the EM data-based path comprises a sequence of points distributed evenly in both spatial and temporal domain and is indicative of a driving trajectory of the tip of the endoscope.

In some embodiments, the body part is a lung and the endoscope is a bronchoscope. In some embodiments, the deformation parameter indicative of a deformation of the body part due to a motion of the patient. In some embodiments, the biological model of the body part of the patient is generated based on a computed tomography (CT) scan. In some embodiments, the deformation parameter is indicative of a deformation due to CT-to-body-divergence.

In some embodiments, the one or more path hypotheses are within a predetermined location range from the latest data point of the sequence of EM data, within a threshold of measurement error or deformation, or above a threshold of the possibility. In some embodiments, each of the one or more path hypotheses comprises at least a portion of a centerline of an airway of the biological model.

In some embodiments, the method further comprises dynamically adding or removing a path hypothesis as the tip of the endoscope is driving through the body part. In some embodiments, mapping the one or more path hypotheses to the EM data-based path comprises applying the optimization algorithm to determine a deformation and shape alignment between the one or more path hypotheses and the EM data-based path. In some embodiments, the deformed path selected from the one or more deformed path paths is associated with the highest probability. In some cases, the highest probability is determined based at least in part normalized probabilities associated with each of the one or more path hypotheses.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example process of localization using deformable path mapping.

FIG. 2 shows an example process of batch localization using deformable path mapping.

FIG. 3 examples of robotic bronchoscopy systems, in accordance with some embodiments of the invention.

FIG. 4 shows an example of a fluoroscopy (tomosynthesis) imaging system.

FIG. 5 and FIG. 6 show examples of a flexible endoscope.

FIG. 7 shows an example of an instrument driving mechanism providing mechanical interface to the handle portion of a robotic bronchoscope.

FIG. 8 shows an example of a distal tip of an endoscope.

FIG. 9 shows an example distal portion of the catheter with integrated imaging device and the illumination device.

FIG. 10 and FIG. 11 show examples of biological models of lungs including EM data and a plurality of localization hypotheses.

FIG. 12 shows a computer system that is programmed or otherwise configured to implement methods provided herein.

FIG. 13 shows an example of a method for presenting one or both of tomosynthesis reconstructions or augmented fluoroscopic overlays.

FIG. 14 shows an exemplary diagram of EM sensor state machine.

FIG. 15 shows an exemplary diagram of registration state machine.

FIG. 16 shows an exemplary diagram of a localization state machine.

### DETAILED DESCRIPTION

While exemplary embodiments will be primarily directed at tomosynthesis, an endoscope (e.g., a bronchoscope), etc., one of skill in the art will appreciate that this is not intended to be limiting, and the systems, methods, and techniques described herein may be used for other therapeutic or diagnostic procedures and in other anatomical regions of a patient's body such as a digestive system, including but not limited to the esophagus, liver, stomach, colon, urinary tract, and various others.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Although the treatment planning and definition of diagnosis or surgical procedures as described herein are presented in the context of pulmonary diagnosis or surgery, the methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) or a microcontroller), digital signal processors (DSPs), a field programmable gate array (FPGA) or one or more Advanced RISC Machine (ARM) processors. In some cases, the one or more processors may be operatively coupled to a non-transitory computer-readable medium. The non-transitory computer-readable medium can store logic, code, or program instructions executable by the one or more processors unit for performing one or more steps. The non-transitory computer-readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus and can be opposite of anatomical references. For example, a distal location of a bronchoscope or catheter may correspond to a proximal location of an elongate member of the patient, and a proximal location of the bronchoscope or catheter may correspond to a distal location of the elongate member of the patient.

A system as described herein, includes an elongate portion or elongate member such as a catheter. The terms “elongate member”, “catheter”, “bronchoscope” are used interchangeably throughout the specification unless contexts suggest otherwise. The elongate member can be placed directly into the body lumen or a body cavity. In some embodiments, the system may further include a support apparatus such as a robotic manipulator (e.g., robotic arm) to drive, support, position or control the movements or operation of the elongate member. Alternatively or in addition to, the support apparatus may be a hand-held device or other control devices that may or may not include a robotic system. In some embodiments, the system may further include peripheral devices and subsystems such as imaging systems that would assist or facilitate the navigation of the elongate member to the target site in the body of a subject. Such navigation may require a registration process which will be described later herein.

In some embodiments of the present disclosure, a robotic bronchoscopy system is provided for performing surgical operations or diagnosis with improved performance at low cost. For example, the robotic bronchoscopy system may comprise a steerable catheter that can be entirely disposable. This may beneficially reduce the requirement of sterilization which can be high in cost or difficult to operate, yet the sterilization or sanitization may not be effective. Moreover, one challenge in bronchoscopy is reaching the upper lobe of the lung while navigating through the airways. In some cases, the provided robotic bronchoscopy system may be designed with capability to navigate through the airway having a small bending curvature in an autonomous or semi-autonomous manner. The autonomous or semi-autonomous navigation may require a registration process. Alternatively, the robotic bronchoscopy system may be navigated by an operator through a control system with vision guidance.

A typical lung cancer diagnosis and surgical treatment process can vary drastically, depending on the techniques used by healthcare providers, the clinical protocols, and the clinical sites. The inconsistent processes may cause delay to diagnose lung cancers in early stage, high cost of healthcare system and the patients to diagnose and treat lung cancers, and high risk of clinical and procedural complications. The robotic bronchoscopy system herein may utilize integrated tomosynthesis to improve lesion visibility and tool-in-lesion confirmation, utilize augmented fluoroscopy allowing for real-time navigation updates and guidance in all areas of the lung, thus allowing for standardized early lung cancer diagnosis and treatment.

In an aspect of the present disclosure, a method is provided for localizing a scope tip of an endoscope in a body part of a patient. The method comprises: (a) obtaining (i) a biological model of the body part of the patient and (ii) electromagnetic (EM) data that is generated by the endoscope;
(b) generating an EM data-based path based on a sequence of the EM data; (c) identifying one or more path hypotheses based on the biological model, and each of the one or more path hypotheses comprises a deformation parameter; (d) determining a probability associated with each of the one or more path hypotheses by mapping the one or more path hypotheses to the EM data-based path; and (e) determining a best fitting path from the one or more path hypotheses based at least in part on the probability associated with each of the one or more path hypotheses as a location for the scope tip. The body part of the patient can include any tissue, organ of the patient such as a lung and/or one or more airways. The EM data-based path is indicative of a trajectory of the scope tip and the EM data are acquired while navigating the scope tip along the one or more airways.

In some cases, the EM data-based path is generated by applying binned filtering and/or time-wise filtering to the EM data. Other suitable processing may be employed so the EM data-based path/trajectory may comprise a sequence of points distributed evenly in both spatial and temporal domain.

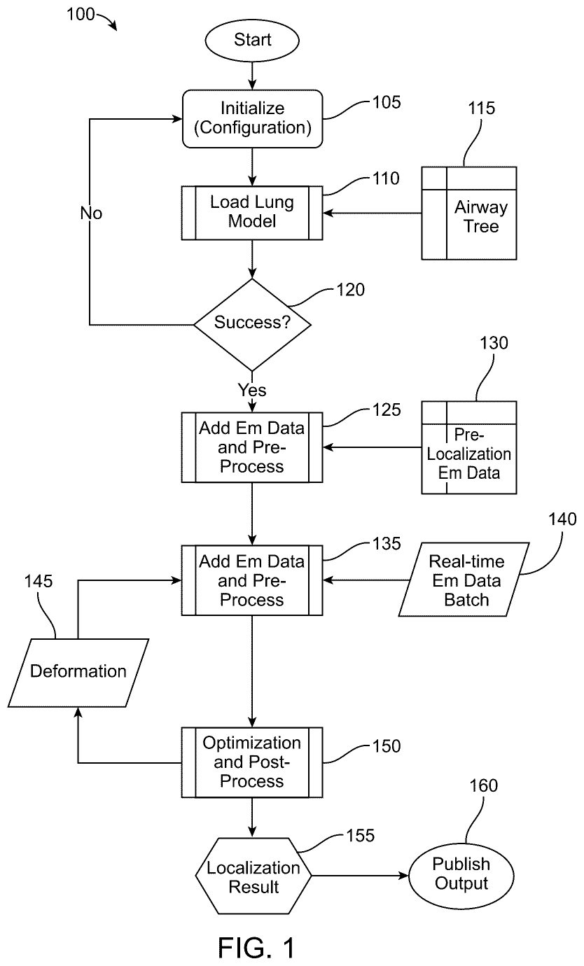

FIG. 1 shows an example process 100 of localization using deformable path mapping. In some cases, the process 100 may comprise localizing a scope tip of a bronchoscope in a body part (e.g., lung) of a patient. The Deformable Path Mapping Localization (DPM Localization) Algorithm or the process 100, which performing the localization with the bronchoscope on the lung, could be applied in any number of ways, such as with other types of endoscopes in other organs or hollow cavities in a body (e.g., human, animal).

The process 100 may begin, in some cases, with initialization (or configuration) at 105. During initialization, various configurations parameters may be obtained. For example, configuration parameters for one or more of EM sensors, registration, localization, localization executor, or navigation may be obtained. Various examples of configurations parameters that may be obtained at the initialization 105.

The input to the DPM algorithm may comprise EM sensor data that is acquired while the scope is traversing/navigated inside of a patient body. The EM sensor data may be acquired at a frequency (e.g., 30 Hz) where the sensor data may contain noise such as Gaussian noise as described above. The output from the DPM algorithm may comprise a localized scope tip position corresponding to the last EM sensor data input.

The DPM algorithm can be implemented by a plurality of functions. In some cases, the process 100 may begin with initializing parameters or configuration of the functions 105. For example, parameters related to the EM sensor data acquisition may be configured (e.g., rate at which to broadcast tracking data, Rate at which to poll for detection of new hardware state, Rate at which to attempt reconnection to hardware, Accepted field generator model identifier, Delay to account for latency when receiving the scope connected contract, etc.), parameters related to registering the EM sensor data with the patient CT scan coordinate system (e.g., State Machine smRegistration) may be configured and such parameters may include, for example, delay between registration update calculations, Scalar describing how much to smooth the incoming tracking signal, Minimum acceptable distance between EM tracking points, Percentage length of the trachea to use for registration point cloud processing, Distance from the target at which EM tracking points will be discarded from the registration point cloud, Minimum amount of buffer from the edge of the field to be used when positioning the field generator and the like, parameters related to the DPM localization and optimization algorithm may be configured and such parameters may include, for example, the max number of threads to use for DPM localization optimization, the max number of hypotheses to keep for DPM localization process (e.g., max number of hypotheses can be set as 2, 3, 4, 5, 6, etc.), Penalty weight base for switching branch of localization, maximum EM data sample in a bin for the bin filtering of the pre-localization data, max number of EM data samples to be processed in a batch for the batch processing of DPM localization and various other parameters.

In some cases, the process 100 may include loading a lung model at 110. The lung model may be generated based on C-arm video data or imaging data acquired using an imaging apparatus such as C-arm imaging system. For example, the lung model may be a 3D model generated based on a CT-scan. The C-arm imaging system may comprise a source (e.g., an X-ray source) and a detector (e.g., an X-ray detector or X-ray imager). In some cases, a single C-arm source may provide video or imaging data at 110. In some cases, different C-arm sources may provide video or imaging data at 110. While any model of an organ or body cavity could be loaded, as illustrated, the lung model is loaded. The lung model may correspond to a patient of interest. The lung model may include an airway tree model 115. As the most basic function of the lung is to bring air and blood in close proximity to allow gas exchange, the airway tree develops adjacent to the arterial vasculature and branches into tightly packed alveoli that may be 100-200 μm in diameter. In some cases, the air tree model may include a geometric model of a patient lung airway tree, including the centerline points, the branch information, and the airway surface mesh.

In some cases, the process 100 may confirm at 120 that the lung model is successfully loaded. If the lung model is not successfully loaded at 120, the process 100 may return to other operations in the process 100, such as the initialization at 105 or display a Flag indicating that the airway tree model is not successfully created and loaded. If the model is successfully loaded at 120, the process 100 may advance to obtaining and pre-processing EM data at 125 from the pre-localization EM data 130. The pre-localization of EM data 130 may comprise adding EM sensor data into an eventQueue continuously. The EM data in the eventQueue may be filtered, converted with EM-to-CT registration and added to DPM localization process.

The EM data may be obtained at 125 one instance. The EM data may be obtained in future instances at 135 as real-time EM data batch at 140. The EM data may be obtained at a plurality of instances, or on a recurring or repeating basis. For example, the EM data may be obtained at a certain frequency, such as 10-60 Hz. When the EM data is obtained at a certain frequency, the EM data may be queued. For example, the EM data may be queued for pre-processing. In another example, the EM data may be queued for localization. The queued EM Sensor Data may be processed as a batch input to the DPM algorithm. For example, a max number of EM data samples may be processed in a batch for the batch processing of DPM localization. The number of the batch value may be 5, 6, 7, 8, 9, 10, 11, 12, 13, 14, 15, 16, 17, 18, 19, 20, and any number of 20 or below 5.

The EM data may include location data corresponding to a tip of a scope. For example, the EM data may include a sequence of data points each corresponding to a location of the tip of the scope, thereby forming a trajectory of the scope's current and previous locations in the EM data. In some cases, the obtaining the EM data from one or more EM sensors on the scope tip may be implemented by a registration state machine (e.g., smEmSensor of FIG. 14). The smEmSensor may be responsible for monitoring and broadcasting information provided by any connected hardware related to EM tracking. This may include a System Control Unit (SCU), System Interface Unit (SIU), Field Generator (FG), and EM sensor. The smEmSensor may publish hardware status information alongside the EM tracking signal data for scope and fiducial sensors. The tracking signal may include a position (represented as a vector) and orientation (represented as a quaternion) describing sensor pose in EM space (global space created by the connected field generator).

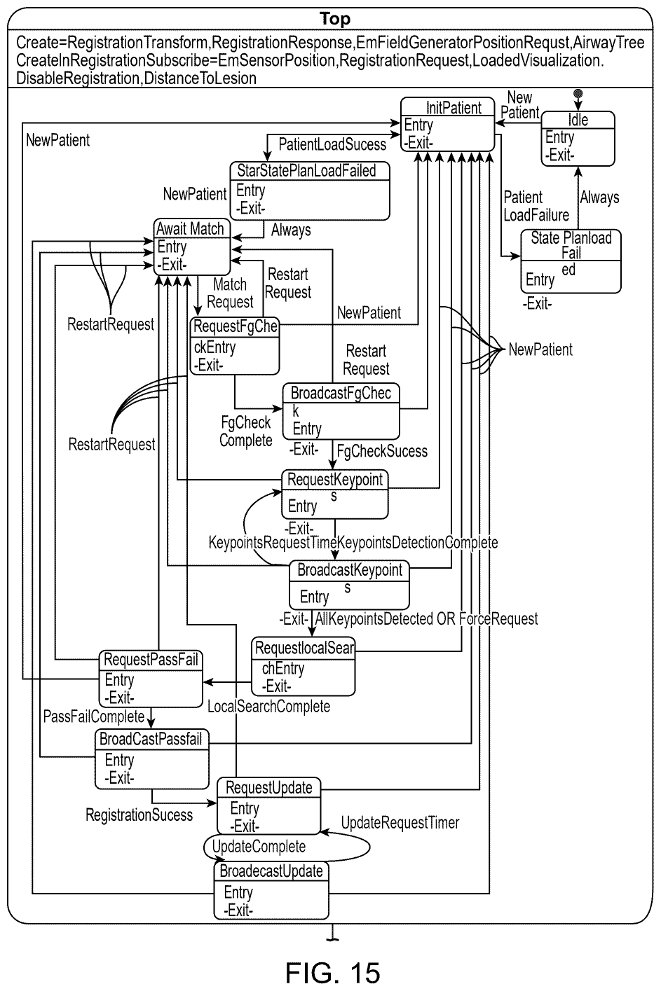

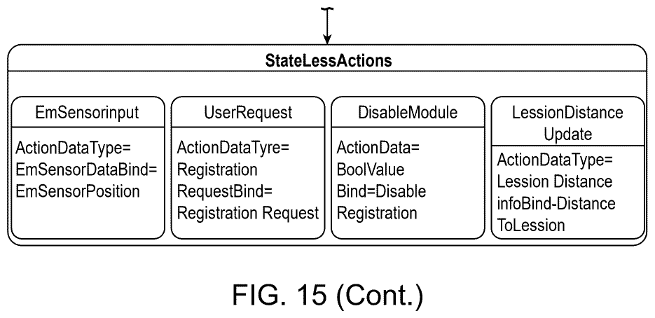

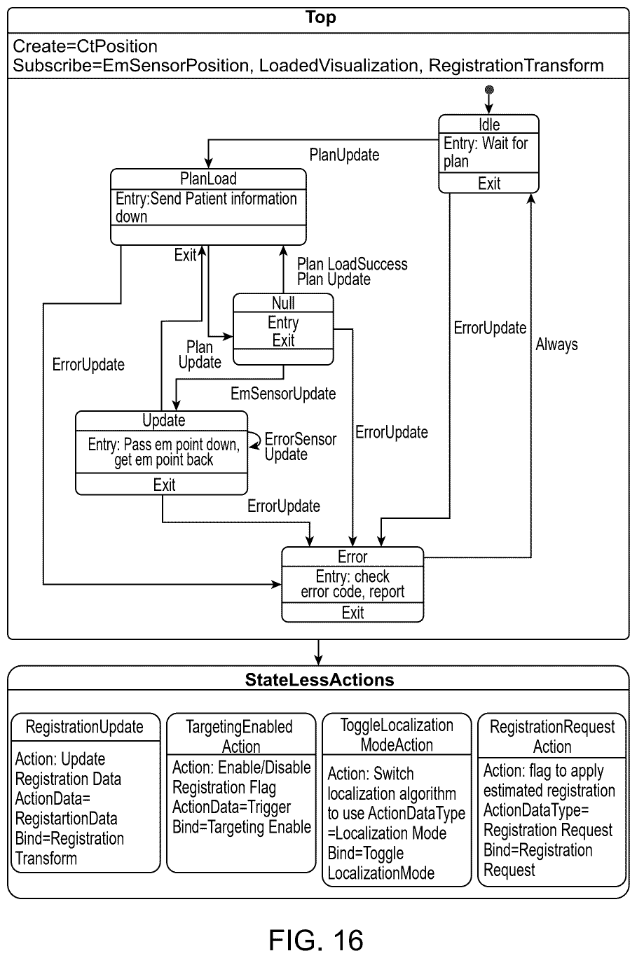

The EM data obtained at 125 and 135 may also be pre-processed. Pre-processing the EM data may include registering the EM data with the CT frame. Transformations that transfer EM sensor data into the CT coordinate frame, may be referred to as EM-To-CT registration. EM-To-CT registration may be published on a dynamic interval. The registering of the EM sensor coordinates to the CT scan coordinates of patient lung may be published as registration transform data. In some cases, registration may include use of a tomosynthesis board. The registration may include use of a modified iterative closest point (ICP) algorithm. At a high level, the registration processes may be illustrated by the registration state machine (smRegistration) of FIG. 15. The smRegistration may be responsible for registering the EM sensor coordinates to the CT scan coordinates of patient lung, then publishing the registration transform data. The registration transform data may then be used by smLocalization (e.g., as illustrated in FIG. 16) to convert EM data to the CT-scan coordinates. The smRegistration send out new updates while the user is in driving mode, away from the lesion.

The EM data may further be processed to generate an EM data-based trajectory or path. In some cases, an EM data-based path may comprise evenly distributed points. For example, the EM data may be processed by applying binned filtering and time-wise filter to evenly distribute the EM data spatially and temporally so to better represent the scope path in a procedure. Alternatively, the points in the EM data-based path may not be evenly distributed.

In some cases, the EM data-based trajectory/path may correspond to a current position of the scope tip. For example, the EM data may be bin filtered, thereby forming a path from the trachea to the position where the last measurement (e.g., EM sensor data) is being processed. In some cases, the EM data beyond the current scope tip position may be omitted, such that in the case of withdrawing the scope, the EM data path beyond may not be accounted for.

In some cases, the process 100 includes performing an optimization and post-processing on the EM data obtained at 125 and 135. The optimization may generate a localization result of an estimated scope tip position. Obtaining the estimated scope tip position may include applying a deformable path mapping (DPM) algorithm to align the scope tip driving trajectory (the EM data sequence) with the lung airway paths. The DPM algorithm takes into account the CT-body divergence and the breathing deformation by modeling the airway paths as deformable hypotheses and applies nonlinear optimization to estimate the deformation and find the best fitting path. To ensure that the DPM algorithm is not blocking the data flow and event process of state machine smLocalization (e.g., as depicted in FIG. 16) processing the computationally intensive DPM process may be done in a separate thread.

In some cases, the DPM algorithm may operate on a plurality of localization or path hypotheses. In some cases, each localization hypothesis may include the centerline points of one or more airways, sequentially from the trachea to the possible scope tip position. In some cases, one or more localization hypotheses may be identified based at least in part on the current scope tip location. For example, one or more localization hypotheses corresponding to one or more airways may be identified within a predetermined range of a current location of the scope tip or EM data-based trajectory while other hypotheses fall outside of the range may be excluded. The one or more identified path hypotheses may be selected as corresponding to the EM data-based trajectory for the following mapping operation.

In some cases, each hypothesis path may be dynamically updated as the EM data is updated or as the scope tip is moving forward or backward. In some cases, one or more localization hypotheses may be dynamically added or removed from a hypothesis pool as the scope tip is driving through the airway. For example, when the scope tip reaches an airway bifurcation, one more localization hypothesis is created to represent the additional possible path that the scope tip can traverse along. In another example, infeasible hypotheses may be removed from the hypothesis pool. For instance, the hypothesis airway path that is far beyond the range of measurement error and deformation, or the hypothesis with a normalized probability that is too small may be removed from the hypothesis pool.

In some cases, each of the localization hypotheses is mapped with the EM data path, solved by computing the mapping of deformable paths by non-linear optimization (e.g., via the software package GMM_Reg). In some cases, the result is the deformed hypothesis path with a deformation parameters 145. For instance, by applying the non-linear optimization algorithm to minimize the displacement energy and the bending energy between each path hypothesis and the EM data path, the path hypothesis may be deformed to fit the EM data path. In some cases, a deformed path with the highest probability may be the final result. For instance, a distal end point on the deformed path may be designated as a location of the scope tip if the scope is moving forward and a proximal end point on the deformed path may be designated as a location of the scope tip if the scope is moving backward. As an example, the highest probability may be calculated such as using a softmax algorithm. The deformed path with the highest probability may be referred to as the most probable path. The deformed path may correspond to a deformation parameter which may be related to deformation of the tissue or organ due to a motion of the patient (e.g., breathing), related to a deformation due to CT-to-body-divergence and/or various other deformation factors. By deforming a path hypothesis to the EM-based path, the deformed path beneficially avoids the real-time CT-to-body-divergence issue or real-time motion of the patient by fitting to the EM-based path.

In some cases, the mapping of the one or more deformable path hypotheses to the EM-based trajectory/path may comprise determining alignment/fitting of the deformable path hypotheses to the EM-based path by solving an optimization problem. For example, for each hypothesis, the average displacement energy and the bending energy may be modeled as Equation 1 and Equation 2, respectively:

$${E}_{p} = {\sum }_{i = 1 , N} {‖ {X}_{EM}^{i} - {X}_{HP}^{i} ‖}^{2} / N , \qquad ( Equation 1 )$$

$${E}_{b} = {\sum }_{i = 1 , N} trace ( {w}^{T} ϕ w ) / N , \qquad ( Equation 2 )$$

where Xⁱ_{EM} is the i-th bin filtered EM data point, Xⁱ_{HP} is the corresponding deformed hypothesis point, w is the K×(D+1) non-affine warping matrix associated with the thin-plate-spline regularization function. ϕ is the K×K matrix formed by the non-affine part of the thin-plate-spline (TPS) deformation model defined by the geometry of the key-points. The combined energy may be modeled as Equation 3:

$${E}_{comb} = λ {E}_{p} + ( 1 - λ ) {E}_{b} . \qquad ( Equation 3 )$$

Based on the combined deformation energy of each hypothesis, the probability may be modeled by Equation 4:

$${P}_{k} = {e}^{{E}_{comb}^{k}} / {\sum }_{k = 1 , M} ( {e}^{{E}_{comb}^{k}} ) , \qquad ( Equation 4 )$$

normalized by

$${\sum }_{k = 1 , M} ( {e}^{{E}_{comb}^{k}} ) ,$$

so that Pₖ of all hypotheses sum up to 1.0, where k is the index of the k'th hypothesis, and M is the number of hypotheses.

In the post-processing at 150, the hypothesis or hypotheses that satisfy a threshold or the path hypothesis with the highest probability may be chosen as the most probable path map, and the corresponding path point or path points that maps to the last EM data position may be output as the localization result at 155. In some cases, satisfying a threshold may include one hypothesis (e.g., the hypothesis with the highest probability) being chosen as the most probable path map. Further, in some cases, the hypotheses are sorted by the probability values from max to min, the last hypotheses beyond the maximum number of hypotheses to keep are pruned.

As an example of implementing the above algorithm, a localization state machine, smLocalization (e.g., as illustrated in FIG. 16) may take the EM data as input, apply the EM-to-CT registration transformation, then estimate the most likely location of the scope tip inside the airways of the patient lung. The localization result may be published at 160 and used for generating the virtual views and for estimating the scope tip distance to lesion. As an example, the data produced by the localization state machine may include the scope tip position data (e.g., including unlocalized position and localized position). Unlocalized position is the scope working channel position which is the EM sensor data converted into CT coordinates by the EM-to-CT transformation. Localized position may correspond to the best estimation of the scope camera position inside the airways computed by the localization algorithm as described above to guide the scope navigation.

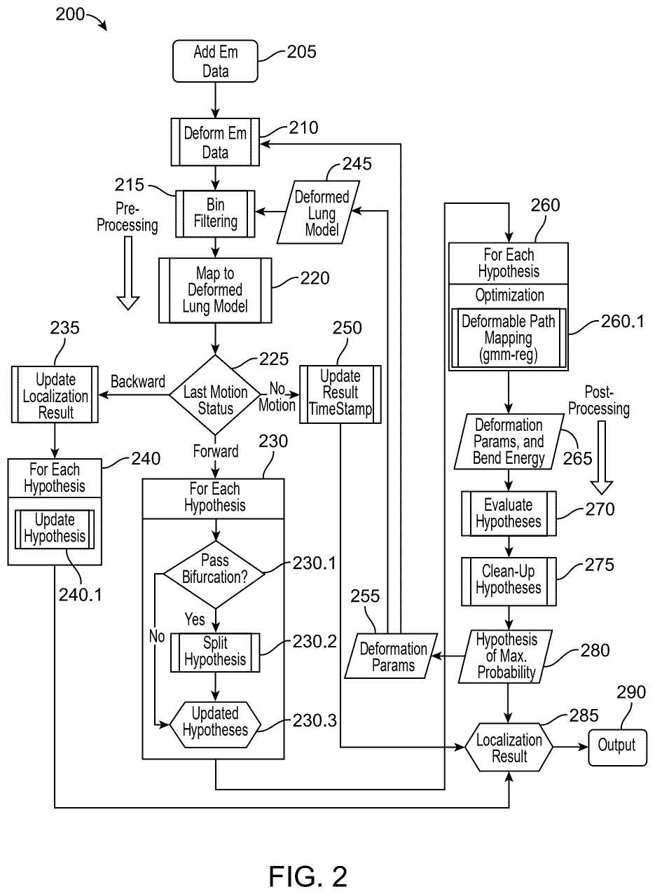

FIG. 2 shows an example process 200 of batch localization using deformable path mapping. The process 200 may be similar to the process 100. While the process 100 provides one example of DMP localization, the process 200 provides an example of batch DPM localization with batch pre-processing optimization and batch post-processing. Some operation of the process 200 may be the same as or similar to operations of the process 100.

For example, the newly acquired EM data may be added 205 to a queue such as by the pre-localization of EM data operation as described above. For instance, the EM data may be added to an eventQueue continuously until reaching a max number of points. The EM data in the eventQueue may be filtered, converted with EM-to-CT registration and added to DPM localization process. In case the DPM localization is slower (with slower processor, or for debugging), the maximum queued size may be set to a number (e.g., 15 by default) that means the DPM localization updates the localization at least once for every maximum (e.g., 15) input EM Sensor Data. When the DPM localization process is fast enough, the queued size may be 1 most of the time. The process may generate an output localized tip position for every such EM Sensor Data input point.

The process may generate deformed EM data 210 based on deformation parameters 255 obtained from the hypothesis with the max probability 280. The deformed EM data may then be bin filtered 215 and generate EM data-based path with points evenly distributed in both temporal and spatial domain. The pre-processing of the EM data may further include mapping the deformed EM-based path to a deformed lung model 220. Next, the process may determine whether the scope tip is moving backward, forward or not moving 225. If no motion is detected, the result timestamp may be updated directly. If the scope tip is detected to be moving backward 235, the localization result may be updated for each path hypothesis 240. The path hypothesis may also be updated by adding or removing new hypothesis or pruning infeasible hypothesis 240.1. if the scope tip is determined to be moving forward 230, the process may proceed to, for each hypothesis, determining whether the scope tip passes a bifurcation 230.1, if yes, then a new hypothesis may be added 230.2, and the hypothesis pool is updated 230.3.

Next, for each path hypothesis 260, the optimization operation is performed to map the deformable hypothesis path to the EM-based path 260.1 (e.g., gmm-reg). The result of the optimization operation may include a deformation parameter and the deformation energy (e.g., bending energy) of each hypothesis 265. Next, the probability for each path hypothesis may be determined based on the deformation parameter and energy 270, and the probabilities may be normalized 275 among all the hypotheses to determine the hypothesis with the maximum probability 280. The path hypothesis with the highest probability may be outputted as the localization result 285 and may be provided to a UI 290 for display.

### Robotic Bronchoscopy System

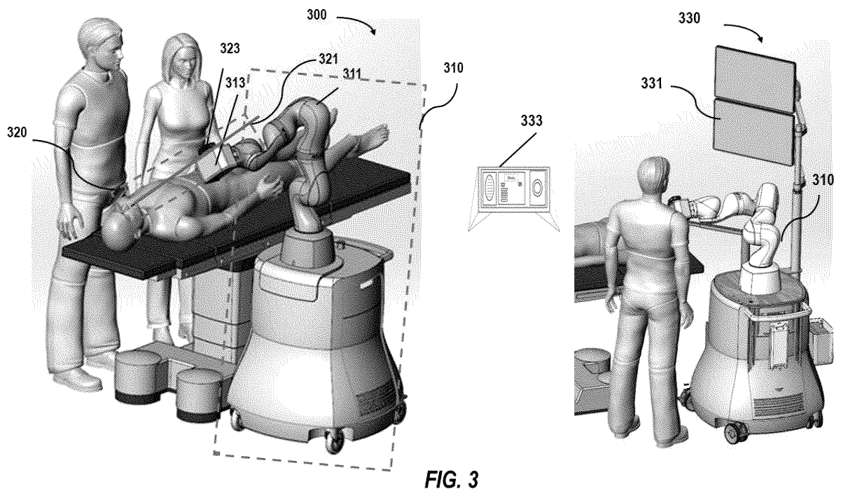

FIG. 3 show examples of robotic bronchoscopy system 300, 330, in accordance with some examples. The robotic bronchoscopy system may implement the systems, the methods, the computer-readable media, and the techniques as described above. For example, the bronchoscopy system 300 may use a scope tip that may be localized using the systems, the methods, the computer-readable media, and the techniques described herein.

As shown in FIG. 3, the robotic bronchoscopy system 300 may comprise a steerable catheter assembly 320 and a robotic support system 310, for supporting or carrying the steerable catheter assembly. The steerable catheter assembly can be a bronchoscope. In some embodiments, the steerable catheter assembly may be a single-use robotic bronchoscope. In some embodiments, the robotic bronchoscopy system 300 may comprise an instrument driving mechanism 313 that is attached to the arm of the robotic support system. The instrument driving mechanism may be provided by any suitable controller device (e.g., hand-held controller) that may or may not include a robotic system. The instrument driving mechanism may provide mechanical and electrical interface to the steerable catheter assembly 320. The mechanical interface may allow the steerable catheter assembly 320 to be releasably coupled to the instrument driving mechanism. For instance, a handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

The steerable catheter assembly 320 may comprise a handle portion 323 that may include components configured to processing image data, provide power, or establish communication with other external devices. For instance, the handle portion 323 may include a circuitry and communication elements that enables electrical communication between the steerable catheter assembly 320 and the instrument driving mechanism 313, and any other external system or devices. In another example, the handle portion 323 may comprise circuitry elements such as power sources for powering the electronics (e.g., camera and LED lights) of the endoscope. In some cases, the handle portion may be in electrical communication with the instrument driving mechanism 313 via an electrical interface (e.g., printed circuit board) so that image/video data or sensor data can be received by the communication module of the instrument driving mechanism and may be transmitted to other external devices/systems. Alternatively or in addition to, the instrument driving mechanism 313 may provide a mechanical interface only. The handle portion may be in electrical communication with a modular wireless communication device or any other user device (e.g., portable/hand-held device or controller) for transmitting sensor data or receiving control signals. Details about the handle portion are described later herein.

The steerable catheter assembly 320 may comprise a flexible elongate member 311 that is coupled to the handle portion. In some embodiments, the flexible elongate member may comprise a shaft, steerable tip and a steerable section. The steerable catheter assembly may be a single use robotic bronchoscope. In some cases, only the elongate member may be disposable. In some cases, at least a portion of the elongate member (e.g., shaft, steerable tip, etc.) may be disposable. In some cases, the entire steerable catheter assembly 320 including the handle portion and the elongate member can be disposable. The flexible elongate member and the handle portion are designed such that the entire steerable catheter assembly can be disposed of at low cost. Details about the flexible elongate member and the steerable catheter assembly are described later herein.

In some embodiments, the provided bronchoscope system may also comprise a user interface. As illustrated in the example system 330, the bronchoscope system may include a treatment interface module 331 (user console side) or a treatment control module 333 (patient and robot side). The treatment interface module may allow an operator or user to interact with the bronchoscope during surgical procedures. In some embodiments, the treatment control module 333 may be a hand-held controller. The treatment control module may, in some cases, comprise a proprietary user input device and one or more add-on elements removably coupled to an existing user device to improve user input experience. For instance, physical trackball or roller can replace or supplement the function of at least one of the virtual graphical element (e.g., navigational arrow displayed on touchpad) displayed on a graphical user interface (GUI) by giving it similar functionality to the graphical element which it replaces. Examples of user devices may include, but are not limited to, mobile devices, smartphones/cellphones, tablets, personal digital assistants (PDAs), laptop or notebook computers, desktop computers, media content players, and the like. Details about the user interface device and user console are described later herein.

The user console 331 may be mounted to the robotic support system 310. Alternatively or in addition to, the user console or a portion of the user console (e.g., treatment interface module) may be mounted to a separate mobile cart.

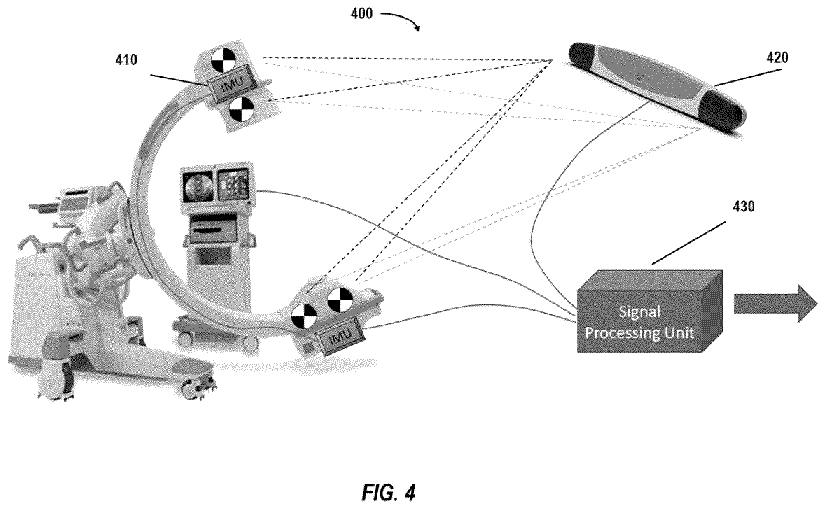

The present disclosure provides a robotic endoluminal platform with integrated tool-in-lesion tomosynthesis technology. In some cases, the robotic endoluminal platform may be a bronchoscopy platform. The platform may be configured to perform one or more operations consistent with the method described herein. FIG. 4 shows an example of a robotic endoluminal platform and its components or subsystems, in accordance with some embodiments of the invention. In some embodiments, the platform may comprise a robotic bronchoscopy system and one or more subsystems that can be used in combination with the robotic bronchoscopy system of the present disclosure.

In some embodiments, the one or more subsystems may include imaging systems such as a fluoroscopy imaging system for providing real-time imaging of a target site (e.g., comprising lesion). Multiple 2D fluoroscopy images may be used to create tomosynthesis or Cone Beam CT (CBCT) reconstruction to better visualize and provide 3D coordinates of the anatomical structures. FIG. 4 shows an example of a fluoroscopy (tomosynthesis) imaging system 400. For example, the fluoroscopy (tomosynthesis) imaging system may perform accurate lesion location tracking or tool-in-lesion confirmation before or during surgical procedure as described above. In some cases, lesion location may be tracked based on location data about the fluoroscopy (tomosynthesis) imaging system/station (e.g., C arm) and image data captured by the fluoroscopy (tomosynthesis) imaging system. The lesion location may be registered with the coordinate frame of the robotic bronchoscopy system.

In some cases, a location, pose or motion of the fluoroscopy imaging system may be measured/estimated to register the coordinate frame of the image to the robotic bronchoscopy system, or for constructing the 3D model/image. The pose or motion of the fluoroscopy (tomosynthesis) imaging system may be measured using any suitable motion/location sensors 410 disposed on the fluoroscopy (tomosynthesis) imaging system. The motion/location sensors may include, for example, inertial measurement units (IMUs)), one or more gyroscopes, velocity sensors, accelerometers, magnetometers, location sensors (e.g., global positioning system (GPS) sensors), vision sensors (e.g., imaging devices capable of detecting visible, infrared, or ultraviolet light, such as cameras), proximity or range sensors (e.g., ultrasonic sensors, lidar, time-of-flight or depth cameras), altitude sensors, attitude sensors (e.g., compasses) or field sensors (e.g., magnetometers, electromagnetic sensors, radio sensors). In some cases, the one or more sensors for tracking the motion and location of the fluoroscopy (tomosynthesis) imaging station may be disposed on the imaging station or be located remotely from the imaging station, such as a wall-mounted camera 420. The C-arm fluoroscopy (tomosynthesis) imaging system in different (rotation) poses while taking images of a subject. The various poses may be captured by the one or more sensors as described above.

In some embodiments, a location of lesion may be segmented in the image data captured by the fluoroscopy (tomosynthesis) imaging system with aid of a signal processing unit 430. One or more processors of the signal processing unit may be configured to further overlay treatment locations (e.g., lesion) on the real-time fluoroscopic image/video. For example, the processing unit may be configured to generate an augmented layer comprising augmented information such as the location of the treatment location or target site. In some cases, the augmented layer may also comprise graphical marker indicating a path to this target site. The augmented layer may be a substantially transparent image layer comprising one or more graphical elements (e.g., box, arrow, etc.). The augmented layer may be superposed onto the optical view of the optical images or video stream captured by the fluoroscopy (tomosynthesis) imaging system, or displayed on the display device. The transparency of the augmented layer allows the optical image to be viewed by a user with graphical elements overlay on top of. In some cases, both the segmented lesion images and an optimum path for navigation of the elongate member to reach the lesion may be overlaid onto the real time tomosynthesis images. This may allow operators or users to visualize the accurate location of the lesion as well as a planned path of the bronchoscope movement. In some cases, the segmented and reconstructed images (e.g., CT images as described elsewhere) provided prior to the operation of the systems described herein may be overlaid on the real time images.

In some embodiments, the one or more subsystems of the platform may comprise one or more treatment subsystems such as manual or robotic instruments (e.g., biopsy needles, biopsy forceps, biopsy brushes) or manual or robotic therapeutical instruments (e.g., RF ablation instrument, Cryo instrument, Microwave instrument, and the like).

In some embodiments, the one or more subsystems of the platform may comprise a navigation and localization subsystem. The navigation and localization subsystem may be configured to construct a virtual airway model based on the pre-operative image (e.g., pre-op CT image or tomosynthesis). The navigation and localization subsystem may be configured to identify the segmented lesion location in the 3D rendered airway model and based on the location of the lesion, the navigation and localization subsystem may generate an optimal path from the main bronchi to the lesions with a recommended approaching angle towards the lesion for performing surgical procedures (e.g., biopsy).

At a registration step before driving the bronchoscope to the target site, the system may align the rendered virtual view of the airways to the patient airways. Image registration may consist of a single registration step or a combination of a single registration step and real-time sensory updates to registration information. The registration process may include finding a transformation that aligns an object (e.g., airway model, anatomical site) between different coordinate systems (e.g., EM sensor coordinates, and patient 3D model coordinates based on pre-operative CT imaging). Details about the registration are described later herein.

Once registered, all airways may be aligned to the pre-operative rendered airways. During robotic bronchoscope driving towards the target site, the location of the bronchoscope inside the airways may be tracked and displayed. In some cases, location of the bronchoscope with respect to the airways may be tracked using positioning sensors. Other types of sensors (e.g., camera) can also be used instead of or in conjunction with the positioning sensors using sensor fusion techniques. Positioning sensors such as electromagnetic (EM) sensors may be embedded at the distal tip of the catheter and an EM field generator may be positioned next to the patient torso during procedure. The EM field generator may locate the EM sensor position in 3D space or may locate the EM sensor position and orientation in 5D or 6D space. This may provide a visual guide to an operator when driving the bronchoscope towards the target site.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter) may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively registered to the patient anatomy (e.g., 3D model) to determine the registration transformation that aligns a single location in the coordinate system with a position in the pre-operative model of the patient's anatomy.

In some embodiments, the platform herein may utilize fluoroscopic imaging systems to determine the location and orientation of medical instruments and patient anatomy within the coordinate system of the surgical environment. In particular, the systems and methods herein may employ a mobile C-arm fluoroscopy as a low-cost and mobile real-time qualitative assessment tool. Fluoroscopy is an imaging modality that obtains real-time moving images of patient anatomy, and medical instruments. Fluoroscopic systems may include C-arm systems which provide positional flexibility and are capable of orbital, horizontal, or vertical movement via manual or automated control. Fluoroscopic image data from multiple viewpoints (i.e., with the fluoroscopic imager moved among multiple locations) in the surgical environment may be compiled to generate two-dimensional or three-dimensional tomographic images. When using a fluoroscopic imager system that include a digital detector (e.g., a flat panel detector), the generated and compiled fluoroscopic image data may permit the sectioning of planar images in parallel planes according to tomosynthesis imaging techniques. The C-arm imaging system may comprise a source (e.g., an X-ray source) and a detector (e.g., an X-ray detector or X-ray imager). The X-ray detector may generate an image representing the intensities of received x-rays. The imaging system may reconstruct 3D image based on multiple 2D image acquired from a wide range of angels. In some cases, the rotation angle range may be at least 120-degree, 130-degree, 140-degree, 150-degree, 160-degree, 170-degree, 180-degree or greater. In some cases, the 3D image may be generated based on a pose of the X-ray imager.

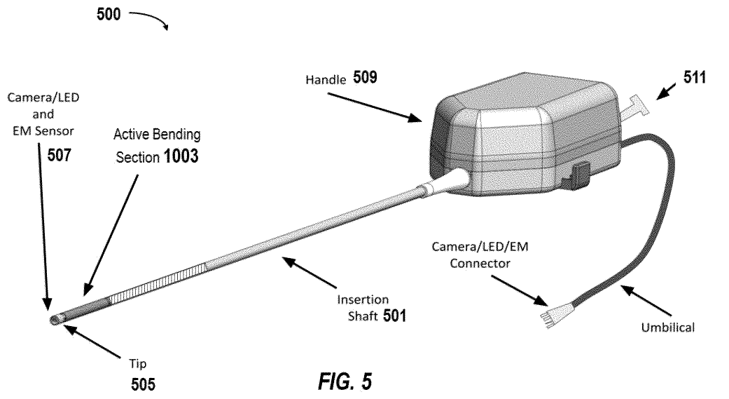

The bronchoscope or the catheter may be disposable. FIG. 5 illustrates an example of a flexible endoscope 500, in accordance with some embodiments of the present disclosure. As shown in FIG. 5, the flexible endoscope 500 may comprise a handle/proximal portion 509 and a flexible elongate member to be inserted inside of a subject. The flexible elongate member can be the same as the one described above. In some embodiments, the flexible elongate member may comprise a proximal shaft (e.g., insertion shaft 501), steerable tip (e.g., tip 505), and a steerable section (active bending section 503). The active bending section, and the proximal shaft section can be the same as those described elsewhere herein. The endoscope 500 may also be referred to as steerable catheter assembly as described elsewhere herein. In some cases, the endoscope 500 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiment, the endoscope may contain varying levels of stiffness along the shaft, as to improve functional operation.

The endoscope or steerable catheter assembly 500 may comprise a handle portion 509 that may include one or more components configured to process image data, provide power, or establish communication with other external devices. For instance, the handle portion may include a circuitry and communication elements that enables electrical communication between the steerable catheter assembly 500 and an instrument driving mechanism (not shown), and any other external system or devices. In another example, the handle portion 509 may comprise circuitry elements such as power sources for powering the electronics (e.g., camera, electromagnetic sensor, and LED lights) of the endoscope.

The one or more components located at the handle may be optimized such that expensive and complicated components may be allocated to the robotic support system, a hand-held controller or an instrument driving mechanism thereby reducing the cost and simplifying the design the disposable endoscope. The handle portion or proximal portion may provide an electrical and mechanical interface to allow for electrical communication and mechanical communication with the instrument driving mechanism. The instrument driving mechanism may comprise a set of motors that are actuated to rotationally drive a set of pull wires of the catheter. The handle portion of the catheter assembly may be mounted onto the instrument drive mechanism so that its pulley/capstans assemblies are driven by the set of motors. The number of pulleys may vary based on the pull wire configurations. In some cases, one, two, three, four, or more pull wires may be utilized for articulating the flexible endoscope or catheter.

The handle portion may be designed allowing the robotic bronchoscope to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cable can be expensive adding to the cost of the bronchoscope. The provided robotic bronchoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

The electrical interface (e.g., printed circuit board) may allow image/video data or sensor data to be received by the communication module of the instrument driving mechanism and may be transmitted to other external devices/systems. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data or receiving control signals.

In some cases, the handle portion 509 may comprise one or more mechanical control modules such as lure 511 for interfacing the irrigation system/aspiration system. In some cases, the handle portion may include lever/knob for articulation control. Alternatively, the articulation control may be located at a separate controller attached to the handle portion via the instrument driving mechanism.

The endoscope may be attached to a robotic support system or a hand-held controller via the instrument driving mechanism. The instrument driving mechanism may be provided by any suitable controller device (e.g., hand-held controller) that may or may not include a robotic system. The instrument driving mechanism may provide mechanical and electrical interface to the steerable catheter assembly 500. The mechanical interface may allow the steerable catheter assembly 500 to be releasably coupled to the instrument driving mechanism. For instance, the handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

In the illustrated example, the distal tip of the catheter or endoscope shaft is configured to be articulated/bent in two or more degrees of freedom to provide a desired camera view or control the direction of the endoscope. As illustrated in the example, imaging device (e.g., camera), position sensors (e.g., electromagnetic sensor) 507 is located at the tip of the catheter or endoscope shaft 505. For example, line of sight of the camera may be controlled by controlling the articulation of the active bending section 503. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with aid of an optimal component.

The distal tip 505 may be a rigid component that allow for positioning sensors such as electromagnetic (EM) sensors, imaging devices (e.g., camera) and other electronic components (e.g., LED light source) being embedded at the distal tip.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. For example, the EM field generator may be positioned close to the patient torso during procedure to locate the EM sensor position in 3D space or may locate the EM sensor position and orientation in 5D or 6D space. This may provide a visual guide to an operator when driving the bronchoscope towards the target site.

The endoscope may have a unique design in the elongate member. In some cases, the active bending section 503, and the proximal shaft of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius).

As described above, the active bending section 503 may be designed to allow for bending in two or more degrees of freedom (e.g., articulation). A greater bending degree such as 180-degrees and 270-degrees (or other articulation parameters for clinical indications) can be achieved by the unique structure of the active bending section. In some cases, a variable minimum bend radius along the axial axis of the elongate member may be provided such that an active bending section may comprise two or more different minimum bend radii.

The articulation of the endoscope may be controlled by applying force to the distal end of the endoscope via one or multiple pull wires. The one or more pull wires may be attached to the distal end of the endoscope. In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle where they are coupled to a driving component (e.g., pulley). This handle pulley may interact with an output shaft from the robotic system.

In some embodiments, the proximal end or portion of one or more pull wires may be operatively coupled to various mechanisms (e.g., gears, pulleys, capstans, etc.) in the handle portion of the catheter assembly. The pull wire may be a metallic wire, cable or thread, or it may be a polymeric wire, cable or thread. The pull wire can also be made of natural or organic materials or fibers. The pull wire can be any type of suitable wire, cable, or thread capable of supporting various kinds of loads without deformation, significant deformation, or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g., SS316), metals, alloys, polymers, nylons, or biocompatible material. Pull wires may be a wire, cable, or a thread. In some embodiments, different pull wires may be made of different materials for varying the load bearing capabilities of the pull wires. In some embodiments, different sections of the pull wires may be made of different material to vary the stiffness or load bearing along the pull. In some embodiments, pull wires may be utilized for the transfer of electrical signals.

The proximal design may improve the reliability of the device without introducing extra cost allowing for a low-cost single-use endoscope. In another aspect of the invention, a single-use robotic endoscope is provided. The robotic endoscope may be a bronchoscope and can be the same as the steerable catheter assembly as described elsewhere herein. Traditional endoscopes can be complex in design and are usually designed to be re-used after procedures, which require thorough cleaning, dis-infection, or sterilization after each procedure. The existing endoscopes are often designed with complex structures to ensure the endoscopes can endure the cleaning, dis-infection, and sterilization processes. The provided robotic bronchoscope can be a single-use endoscope that may beneficially reduce cross-contamination between patients and infections. In some cases, the robotic bronchoscope may be delivered to the medical practitioner in a pre-sterilized package and are intended to be disposed of after a single-use.

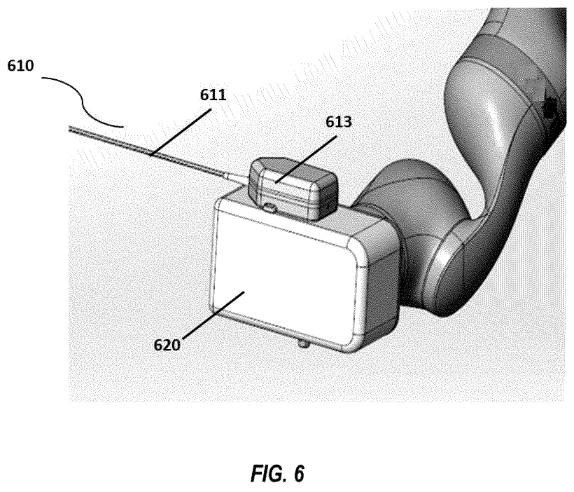

As shown in FIG. 6, a robotic bronchoscope 610 may comprise a handle portion 613 and a flexible elongate member 611. In some embodiments, the flexible elongate member 611 may comprise a shaft, steerable tip, and a steerable/active bending section. The robotic bronchoscope 610 can be the same as the steerable catheter assembly as described in FIG. 5. The robotic bronchoscope may be a single-use robotic endoscope. In some cases, only the catheter may be disposable. In some cases, at least a portion of the catheter may be disposable. In some cases, the entire robotic bronchoscope may be released from the instrument driving mechanism and can be disposed of. In some cases, the bronchoscope may contain varying levels of stiffness along its shaft, as to improve functional operation. In some cases, a minimum bend radius along the shaft may vary.

The robotic bronchoscope can be releasably coupled to an instrument driving mechanism 620. The instrument driving mechanism 620 may be mounted to the arm of the robotic support system or to any actuated support system as described elsewhere herein. The instrument driving mechanism may provide mechanical and electrical interface to the robotic bronchoscope 610. The mechanical interface may allow the robotic bronchoscope 610 to be releasably coupled to the instrument driving mechanism. For instance, the handle portion of the robotic bronchoscope can be attached to the instrument driving mechanism via quick install/release means, such as magnets and spring-loaded levels. In some cases, the robotic bronchoscope may be coupled or released from the instrument driving mechanism manually without using a tool.

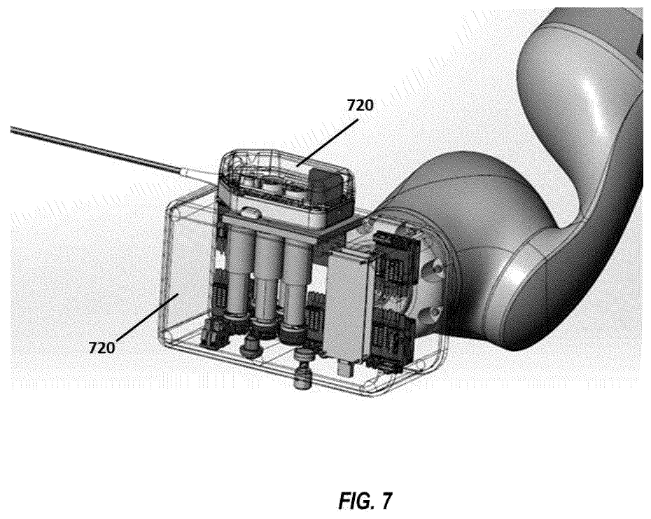

FIG. 7 shows an example of an instrument driving mechanism 700B providing mechanical interface to the handle portion 713 of the robotic bronchoscope. As shown in the example, the instrument driving mechanism 700B may comprise a set of motors that are actuated to rotationally drive a set of pull wires of the flexible endoscope or catheter. The handle portion 713 of the catheter assembly may be mounted onto the instrument drive mechanism so that its pulley assemblies or capstans are driven by the set of motors. The number of pulleys may vary based on the pull wire configurations. In some cases, one, two, three, four, or more pull wires may be utilized for articulating the flexible endoscope or catheter.

The handle portion may be designed allowing the robotic bronchoscope to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cable can be expensive, adding to the cost of the bronchoscope. The provided robotic bronchoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

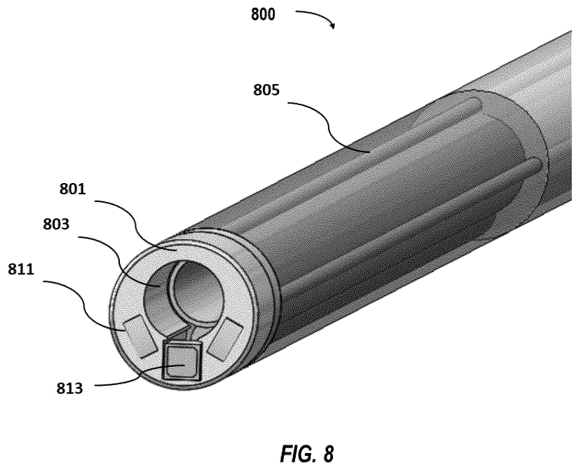

FIG. 8 shows an example of a distal tip 800 of an endoscope. In some cases, the distal portion or tip of the catheter 800 may be substantially flexible such that it can be steered into one or more directions (e.g., pitch, yaw). The catheter may comprise a tip portion, bending section, and insertion shaft. In some embodiments, the catheter may have variable bending stiffness along the longitudinal axis direction. For instance, the catheter may comprise multiple sections having different bending stiffness (e.g., flexible, semi-rigid, and rigid). The bending stiffness may be varied by selecting materials with different stiffness/rigidity, varying structures in different segments (e.g., cuts, patterns), adding additional supporting components or any combination of the above. In some embodiments, the catheter may have variable minimum bend radius along the longitudinal axis direction. The selection of different minimum bend radius at different location long the catheter may beneficially provide anti-prolapse capability while still allow the catheter to reach hard-to-reach regions. In some cases, a proximal end of the catheter needs not be bent to a high degree thus the proximal portion of the catheter may be reinforced with additional mechanical structure (e.g., additional layers of materials) to achieve a greater bending stiffness. Such design may provide support and stability to the catheter. In some cases, the variable bending stiffness may be achieved by using different materials during extrusion of the catheter. This may advantageously allow for different stiffness levels along the shaft of the catheter in an extrusion manufacturing process without additional fastening or assembling of different materials.

The distal portion of the catheter may be steered by one or more pull wires 805. The distal portion of the catheter may be made of any suitable material such as co-polymers, polymers, metals, or alloys such that it can be bent by the pull wires. In some embodiments, the proximal end or terminal end of one or more pull wires 805 may be coupled to a driving mechanism (e.g., gears, pulleys, capstan etc.) via the anchoring mechanism as described above.

The pull wire 805 may be a metallic wire, cable, or thread, or it may be a polymeric wire, cable, or thread. The pull wire 805 can also be made of natural or organic materials or fibers. The pull wire 805 can be any type of suitable wire, cable, or thread capable of supporting various kinds of loads without deformation, significant deformation, or breakage. The distal end or portion of one or more pull wires 805 may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The catheter may have a dimension so that one or more electronic components can be integrated to the catheter. For example, the outer diameter of the distal tip may be around 4 to 4.4 millimeters (mm), and the diameter of the working channel may be around 2 mm such that one or more electronic components can be embedded into the wall of the catheter. However, it should be noted that based on different applications, the outer diameter can be in any range smaller than 4 mm or greater than 4.4 mm, and the diameter of the working channel can be in any range according to the tool dimensional or specific application.

The one or more electronic components may comprise an imaging device, illumination device or sensors. In some embodiments, the imaging device may be a video camera 813. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 811 positioned at the distal tip. The light source may be a light-emitting diode (LED), an organic LED (OLED), a quantum dot, or any other suitable light source. In some cases, the light source may be miniaturized LED for a compact design or Dual Tone Flash LED Lighting.

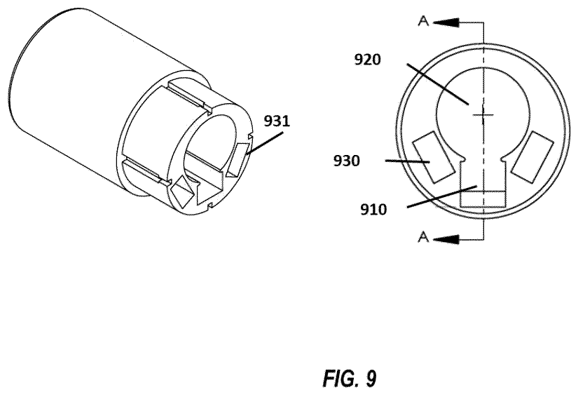

The imaging device and the illumination device may be integrated to the catheter. For example, the distal portion of the catheter may comprise suitable structures matching at least a dimension of the imaging device and the illumination device. The imaging device and the illumination device may be embedded into the catheter. FIG. 9 shows an example distal portion of the catheter with integrated imaging device and the illumination device. A camera may be located at the distal portion. The distal tip may have a structure to receive the camera, illumination device or the location sensor. For example, the camera may be embedded into a cavity 910 at the distal tip of the catheter. The cavity 910 may be integrally formed with the distal portion of the cavity and may have a dimension matching a length/width of the camera such that the camera may not move relative to the catheter. The camera may be adjacent to the working channel 920 of the catheter to provide near field view of the tissue or the organs. In some cases, the attitude or orientation of the imaging device may be controlled by controlling a rotational movement (e.g., roll) of the catheter.

The power to the camera may be provided by a wired cable. In some cases, the cable wire may be in a wire bundle providing power to the camera as well as illumination elements or other circuitry at the distal tip of the catheter. The camera or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light may be provided by fiber cables that transfer the light of a light source located at the proximal end of the endoscope, to the distal end of the robotic endoscope. In some embodiments of the disclosure, miniaturized LED lights may be employed and embedded into the distal portion of the catheter to reduce the design complexity. In some cases, the distal portion may comprise a structure 930 having a dimension matching a dimension of the miniaturized LED light source. As shown in the illustrated example, two cavities 530 may be integrally formed with the catheter to receive two LED light sources. For instance, the outer diameter of the distal tip may be around 4 to 4.4 millimeters (mm) and diameter of the working channel of the catheter may be around 2 mm such that two LED light sources may be embedded at the distal end. The outer diameter can be in any range smaller than 4 mm or greater than 4.4 mm, and the diameter of the working channel can be in any range according to the tool's dimensional or specific application. Any number of light sources may be included. The internal structure of the distal portion may be designed to fit any number of light sources.

In some cases, each of the LEDs may be connected to power wires which may run to the proximal handle. In some embodiment, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover 931 may be placed at the forwarding end face of the distal tip providing precise positioning of the LEDs as well as sufficient room for the glue. The cover 931 may be composed of transparent material matching the refractive index of the glue so that the illumination light may not be obstructed.

### Examples of Localization Hypotheses

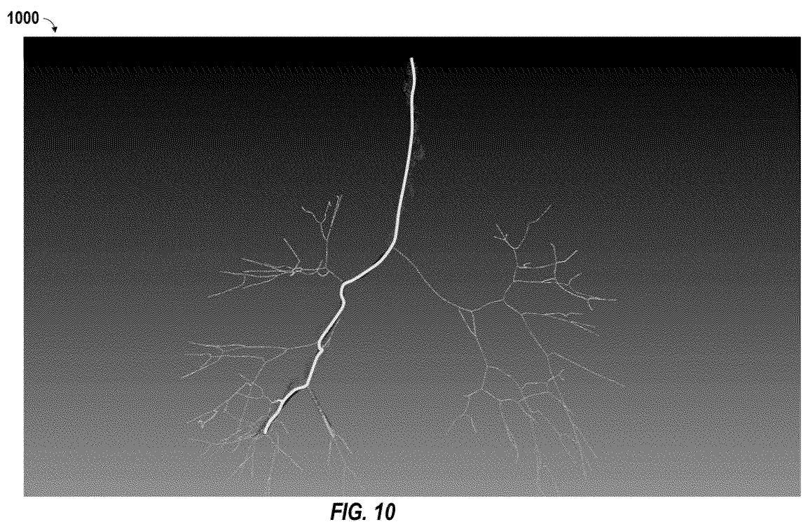

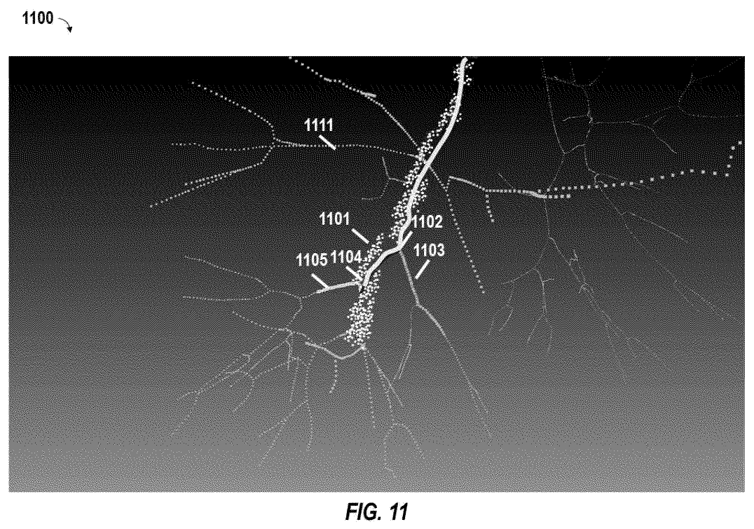

FIG. 10 and FIG. 11 show examples 1000 and 1100, respectively, of biological models of lungs including EM data and a plurality of localization hypotheses. The examples 1000 and 1100 may be, in some cases, depicting an internal computational process. The examples 1000 and 1100 may be, in some cases, presented, in whole or in part, on a graphical display. The examples 1000 and 1100 may, in some cases, represent the processes 100 or 200. The examples 1000 and 1100 may, in some cases, represent the hardware, software, and techniques described with respect to FIGS. 3-9 (e.g., endoscopy, bronchoscopy, etc.).

Each of the examples 1000 and 1100 include a lung model. The lung model may be generated based on a CT-scan. EM data is also depicted in the examples 1000 and 1100. The EM data may be obtained using EM sensors on a scope tip (e.g., a bronchoscope tip). As illustrated, the EM data may be registered, as in, the EM data may be translated to the same coordinate set as the lung model of the CT-scan. The examples 1000 and 1100 show a plurality of localization hypotheses, each corresponding to a different possible path down the lung model, based on the EM data. For instance, a localization hypothesis or a path hypothesis 1000, 1100 may have a shape based on a segment of centerline of an airway where the centerline is determined based on the CT-scan, and a location of the path hypothesis may be based on the registration. As shown, the plurality of localization hypotheses are within a certain distance range from the EM data. In particular, as illustrated, the EM data is within a certain distance from the latest points of the EM data. In doing so, the examples 1000 and 1100 may avoid hypotheses 1111 that are highly improbable due to how far away they are from the latest points of the EM data.

As illustrated in the example 1100, the deformable path hypothesis may be dynamically added or removed as the scope tip traversing along the airways. For example, when the scope tip reaches a first bifurcation 1102 (scope trajectory 1101), a new hypothesis 1103 may be added. When the scope tip reaches a second bifurcation 1104, a new hypothesis 1105 may be added. Other hypotheses 1111 fall outside of a predetermined distance range of the scope tip or an EM data point may be excluded.

At a high level, the examples 1000 and 1100 may implement deformable path mapping (DPM) operations of (A) modeling the EM data as a path that is registered; (B) applying binned filtering and time-wise filter to evenly distribute the EM data spatially and temporally (e.g., so to better represent the scope path in a procedure); (C) formatting all possible airway paths in the lung model within a certain range (e.g., distance) as possible matches to the EM data, which may be referred to as localization hypotheses; (D) creating more localization hypotheses as the scope drives further and the EM data is updated (e.g., when the scope reaches an airway bifurcation, one more localization hypothesis is created to represent the two possibilities); (E) mapping the airway path of each localization hypothesis with the EM data, where the mapping is formed as an optimization problem that solves the deformation and shape alignment; (F) solving the optimization problem to estimate the deformation and shape alignment mapping for each of the localization hypotheses; (G) estimating the probability of each of the localization hypotheses based on the deformation and shape alignment mapping results (e.g., using normalized probabilities of an existing hypothesis to determine the best solution of the mapping); (H) outputting the localization hypothesis with the highest probability; (I) pruning off infeasible localization hypotheses (e.g., localization hypotheses that are far beyond the range of measurement error and deformation, or localization hypotheses with a normalized probability below a threshold); (J) passing the remaining (non-pruned) localization hypotheses to progressively continue being evaluated using updated EM data obtained as the scope continues to move further through the patient. In some cases, as the DPM operations are performed and solved in real-time, dynamic deformations, such as breathing motion, may be accounted for. In some cases, the EM data and airways of the lungs may be modeled as Gaussian mixture models. In some cases, the DPM optimization is solved as an optimization problem in combination with proper regularization functions. In some cases, the optimization is solved as a nonlinear optimization. In some cases, the binned filtering of the EM data helps to obtain clean and well-distributed paths. In some cases, the DPM operations may account for CT-to-body-divergence. In some cases, the DPM operations can be applied to localization with other sensors, such as scope shape sensing technologies.

### Computer System

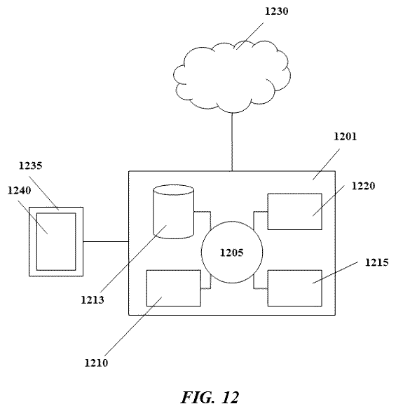

The present disclosure provides computer systems that are programmed to implement the methods, the computer-readable media, and the techniques of the disclosure. FIG. 12 shows a computer system 1201 that is programmed or otherwise configured to operate the methods, the computer-readable media, and the techniques described herein (such as methods of localizing a scope tip of an endoscope in an organ of a patient, described herein). For example, the computer system 1201 may implement the processes 100 or 200. In another example, the user interface 1240 may present one or more of the example models described with respect to FIG. 10 or 11.

The computer system 1201 can regulate various aspects of the present disclosure, such as, for example, techniques for localizing a scope tip of an endoscope in an organ of a patient. The computer system 1201 can be an electronic device of a user or a computer system that is remotely located with respect to the electronic device. The electronic device can be a mobile electronic device.

The computer system 1201 includes a central processing unit (CPU, also “processor” and “computer processor” herein) 1205, which can be a single core or multi core processor, or a plurality of processors for parallel processing. The computer system 1201 also includes memory or memory location 1210 (e.g., random-access memory, read-only memory, flash memory), electronic storage unit 1215 (e.g., hard disk), communication interface 1220 (e.g., network adapter) for communicating with one or more other systems, and peripheral devices 1225, such as cache, other memory, data storage or electronic display adapters. The memory 1210, storage unit 1215, interface 1220 and peripheral devices 1225 are in communication with the CPU 1205 through a communication bus (solid lines), such as a motherboard. The storage unit 1215 can be a data storage unit (or data repository) for storing data. The computer system 1201 can be operatively coupled to a computer network (“network”) 1230 with the aid of the communication interface 1220. The network 1230 can be the Internet, an internet or extranet, or an intranet or extranet that is in communication with the Internet. The network 1230 in some cases is a telecommunication or data network. The network 1230 can include one or more computer servers, which can enable distributed computing, such as cloud computing. The network 1230, in some cases with the aid of the computer system 1201, can implement a peer-to-peer network, which may enable devices coupled to the computer system 1201 to behave as a client or a server.

The CPU 1205 can execute instructions on computer-readable media, which can be embodied in a program or software. The instructions may be stored in a memory location, such as the memory 1210. The instructions can be directed to the CPU 1205, which can subsequently program or otherwise configure the CPU 1205 to implement methods of the present disclosure. Examples of operations performed by the CPU 1205 can include fetch, decode, execute, and writeback.

The CPU 1205 can be part of a circuit, such as an integrated circuit. One or more other components of the system 1201 can be included in the circuit. In some cases, the circuit is an application specific integrated circuit (ASIC).

The storage unit 1215 can store files, such as drivers, libraries, and saved programs. The storage unit 1215 can store user data, e.g., user preferences and user programs. The computer system 1201 in some cases can include one or more additional data storage units that are external to the computer system 1201, such as located on a remote server that is in communication with the computer system 1201 through an intranet or the Internet.

The computer system 1201 can communicate with one or more remote computer systems through the network 1230. For instance, the computer system 1201 can communicate with a remote computer system of a user (e.g., a medical device operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1201 via the network 1230.

Methods as described herein can be implemented by way of machine (e.g., computer processor) executable code stored on an electronic storage location of the computer system 1201, such as, for example, on the memory 1210 or electronic storage unit 1215. The instructions may be code stored on the computer-readable media can be provided in the form of software. During use, the code can be executed by the processor 1205. In some cases, the code can be retrieved from the storage unit 1215 and stored on the memory 1210 for ready access by the processor 1205. In some situations, the electronic storage unit 1215 can be precluded, and machine-executable instructions are stored on memory 1210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1201, can be embodied in programming. Various aspects of the technology may be thought of as “products” or “articles of manufacture” typically in the form of computer-readable media storing instructions as code or associated data that is carried on or embodied in a type of computer-readable media. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable media” refer to any medium or media that participates in providing instructions to a processor for execution.

Hence, a computer-readable media, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code or data. Many of these forms of computer-readable media may be involved in carrying one or more sequences of one or more instructions to a processor for execution.

The computer system 1201 can include or be in communication with an electronic display 1235 that comprises a user interface (UI) 1240 for providing, for example, for tomosynthesis (e.g., tomosynthesis reconstruction) or fluoroscopy (e.g., augmented fluoroscopy) data, such as text, video, images, etc. Examples of UI's include, without limitation, a graphical user interface (GUI), a web-based user interface, or an Application Programming Interface (API). The UI 1240 may be, in some cases, also used for input via touchscreen capabilities.

Methods, systems, instructions, and techniques of the present disclosure can be implemented by way of one or more algorithms. An algorithm can be implemented by way of software upon execution by the central processing unit 1205. The algorithm can, for example, (a) obtain (i) a biological model of an organ of a patient and (ii) electromagnetic (EM) data that is generated by an endoscope in the organ; (b) generate a plurality of localization hypotheses for the scope tip based on the biological model and the EM data; (c) generate a plurality of deformations, wherein each deformation of the plurality of deformations respectively maps each of the plurality of localization hypotheses for the scope tip to the EM data; (d) determine a localization for the scope tip from the plurality of localization hypotheses for the scope tip, wherein the localization corresponds to a predicted deformation of the plurality of deformations that satisfies a threshold; and (e) cause the localization for the scope tip to be presented on a graphical display.

### Example Method

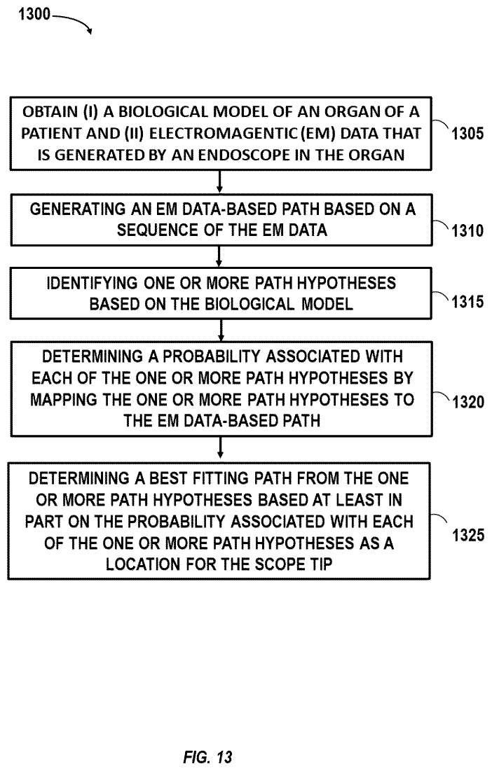

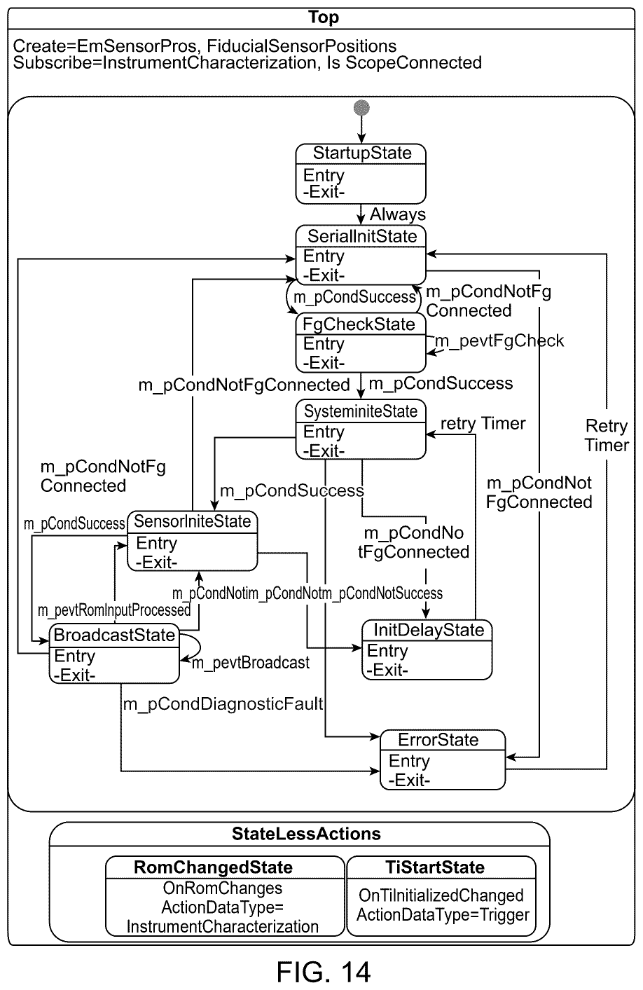

FIG. 13 illustrates an example method 1300 of localizing a scope tip of an endoscope in an organ of a patient, consistent with certain examples of the present disclosure. The method may include obtaining (i) a biological model of the body part of the patient and (ii) electromagnetic (EM) data that is generated by the endoscope (operation 1305); generating an EM data-based path based on a sequence of the EM data (operation 1310); identifying one or more path hypotheses based on the biological model, wherein each of the one or more path hypotheses comprises a deformation parameter (operation 1315); determining a probability associated with each of the one or more path hypotheses by mapping the one or more path hypotheses to the EM data-based path (operation 1320); determining a best fitting path from the one or more path hypotheses based at least in part on the probability associated with each of the one or more path hypotheses as a location for the scope tip (operation 1325). The method 1300 may implement one or more of the systems, the computer-readable media, the techniques, or the like that are described herein.

In some cases, the method 1300 may begin with obtaining (i) the biological model of the organ of the patient and (ii) the EM data that is generated by the endoscope in the organ at the block 1305. In some cases, the organ is a lung and the endoscope is a bronchoscope. In some cases, the biological model of the organ of the patient is generated by a computed tomography (CT) scan.

In some cases, the method 1300 may include generating the plurality of localization hypotheses for the scope tip based on the biological model and the EM data at block 1310. In some cases, prior to block 1310, the method 1300 may include translating the EM data into a coordinate frame of the biological model (e.g., as described with respect to smRegistration). In some cases, each of the localization hypotheses includes a predicted path (e.g., as depicted in FIGS. 10 and 11) for the scope tip though the organ. In some cases, each of the localization hypotheses includes a predicted location for the scope tip in the organ. In some cases, each of the localization hypotheses includes a predicted orientation for the scope tip in the organ. In some cases, each of the plurality of hypotheses are within a range of a latest data point of the EM data. In some cases, the range may be a predetermined number that is determined based on empirical data. In some cases, the range may be configurable by a user.

In some cases, the method 1300 may include generating the plurality of deformations, wherein each deformation of the plurality of deformations respectively maps each of the plurality of localization hypotheses for the scope tip to the EM data at the block 1315. In some cases, the plurality of deformations comprise deformation of the lung due to the patient breathing. In some cases, the plurality of deformations comprise deformation due to CT-to-body-divergence.

In some cases, the method 1300 may include determining the localization for the scope tip from the plurality of localization hypotheses for the scope tip, wherein the localization corresponds to the predicted deformation of the plurality of deformations that satisfies the threshold at the block 1320. In some cases, prior to block 1320, the method 1300 may include generating a plurality of probability scores corresponding to the plurality of deformations. In some cases, the threshold is based on each probability score of the plurality of probability scores that respectively corresponds to each deformation of the plurality of deformations. In some cases, a probability score of the predicted deformation corresponding to the localization for the scope tip satisfies the threshold based on the probability score being greater than every other probability score of the plurality of probability scores.

In some cases, the method 1300 may include causing the localization for the scope tip to be presented on the graphical display at the block 1325. In some cases, the localization for the scope tip is superimposed on the biological model of the organ when presented on the graphical display. In some cases, the method 1300 may further comprise determining a distance of the scope tip to a lesion in the organ based on the localization. In some cases, the method 1300 may further comprise (i) updating, by the one or more processors, the EM data to include new electromagnetic (EM) data, wherein the new EM data is generated by the endoscope; and (ii) perform operations corresponding to the blocks 1310, 1315, 1320, and 1325 using the updated EM data. This may continue repeatedly as updated EM data is obtained. For example, if updated EM data is obtained at a frequency of 30 Hz, then the operations corresponding to the blocks 1310, 1315, 1320, and 1325 may be performed at a frequency of 30 Hz using the updated EM data. In another example, if updated EM data is obtained at a frequency of 30 Hz, then the operations corresponding to the blocks 1310, 1315, 1320, and 1325 may be performed at a frequency of less than 30 Hz (e.g., five times per second, once per second, ten times per minute, etc.) using the updated EM data.

### Additional Considerations

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

It should be understood, that any reference herein to the term “or” is intended to mean an “inclusive or” or what is also known as a “logical OR”, wherein when used as a logic statement, the expression “A or B” is true if either A or B is true, or if both A and B are true, and when used as a list of elements, the expression “A, B or C” is intended to include all combinations of the elements recited in the expression, for example, any of the elements selected from the group consisting of A, B, C, (A, B), (A, C), (B, C), and (A, B, C); and so on if additional elements are listed. Furthermore, it should also be understood that the indefinite articles “a” or “an”, and the corresponding associated definite articles “the” or “said”, are each intended to mean one or more unless otherwise stated, implied, or physically impossible. Yet further, it should be understood that the expressions “at least one of A and B, etc.”, “at least one of A or B, etc.”, “selected from A and B, etc.” and “selected from A or B, etc.” are each intended to mean either any recited element individually or any combination of two or more elements, for example, any of the elements from the group consisting of “A”, “B”, and “A AND B together”, etc.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where values are described in the application and claims, unless otherwise stated the term “about” meaning within an acceptable error range for the particular value may be assumed.

It should be noted that various illustrative or suggested ranges set forth herein are specific to their example embodiments and are not intended to limit the scope or range of disclosed technologies, but, again, merely provide example ranges for frequency, amplitudes, etc. associated with their respective embodiments or use cases.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method for localizing an endoscope in a body part of a patient, the method comprising:
(a) obtaining a sequence of electromagnetic (EM) data;
(b) generating an EM data-based path based at least in part on the sequence of the EM data;
(c) identifying one or more path hypotheses based at least in part on a data point from the sequence of EM data, wherein the one or more path hypotheses have a shape based on a biological model of the body part of the patient;
(d) generating one or more deformed paths by mapping the one or more path hypotheses to the EM data-based path using an optimization algorithm; and
(e) selecting a deformed path from the one or more deformed paths based at least in part on a probability associated with each of the one or more path hypotheses and determining a location for a tip of the endoscope based on the selected deformed path.

2. The method of claim 1, wherein, prior to (c), the EM data-based path is translated into a coordinate frame of the biological model.

3. The method of claim 1, wherein the biological model comprises one or more airways.

4. The method of claim 3, wherein the EM data are acquired while navigating the tip of the endoscope along the one or more airways forward or backward.

5. The method of claim 1, wherein the EM data-based path is generated by applying binned filtering and/or time-wise filtering to the EM data.

6. The method of claim 5, wherein the EM data-based path comprises a sequence of points distributed evenly in both spatial and temporal domain and is indicative of a driving trajectory of the tip of the endoscope.

7. The method of claim 1, wherein the body part is a lung and the endoscope is a bronchoscope.

8. The method of claim 1, wherein the deformation parameter indicative of a deformation of the body part due to a motion of the patient.

9. The method of claim 1, wherein the biological model of the body part of the patient is generated based on a computed tomography (CT) scan.

10. The method of claim 1, wherein the deformation parameter is indicative of a deformation due to CT-to-body-divergence.

11. The method of claim 1, wherein the one or more path hypotheses are within a predetermined location range from the latest data point of the sequence of EM data, within a threshold of measurement error or deformation, or above a threshold of the possibility.

12. The method of claim 1, wherein each of the one or more path hypotheses comprises at least a portion of a centerline of an airway of the biological model.

13. The method of claim 1, further comprising dynamically adding or removing a path hypothesis as the tip of the endoscope is driving through the body part.

14. The method of claim 1, wherein mapping the one or more path hypotheses to the EM data-based path comprises applying the optimization algorithm to determine a deformation and shape alignment between the one or more path hypotheses and the EM data-based path.

15. The method of claim 1, wherein the deformed path selected from the one or more deformed path paths is associated with the highest probability.

16. The method of claim 15, wherein the highest probability is determined based at least in part normalized probabilities associated with each of the one or more path hypotheses.

17. A system for localizing an endoscope in a body part of a patient, the system comprising: a memory storing computer-executable instructions; one or more processors in communication with the endoscope and configured to execute the computer-executable instructions to:
(a) obtain a sequence of electromagnetic (EM) data;
(b) generate an EM data-based path based at least in part on the sequence of the EM data;
(c) identify one or more path hypotheses based at least in part on a data point from the sequence of EM data, wherein the one or more path hypotheses have a shape based on a biological model of the body part of the patient;
(d) generate one or more deformed paths by mapping the one or more path hypotheses to the EM data-based path using an optimization algorithm; and
(e) select a deformed path from the one or more deformed paths based at least in part on a probability associated with each of the one or more path hypotheses and determining a location for a tip of the endoscope based on the selected deformed path.

18. The system of claim 17, wherein, prior to (c), the EM data-based path is translated into a coordinate frame of the biological model.

19. The system of claim 17, wherein the biological model comprises one or more airways.

20. The system of claim 19, wherein the EM data are acquired while navigating the tip of the endoscope along the one or more airways forward or backward
